(19) United States
(12) Patent Application Publication
(10) Pub. No.: US 2020/0268256 A1
(43) Pub. Date: Aug. 27, 2020

(54) INJECTABLE BIOCOMPATIBLE SENSOR SYSTEM FOR MEASURING AND COMMUNICATING PHYSIOLOGICAL DATA

(71) Applicant: Medtronic, Inc., Minneapolis, MN (US)

(72) Inventors: William C. Harding, Chandler, AZ (US); Martha De Cunha Maluf-Burgman, The Hague (NL)

(21) Appl. No.: 16/286,956

(22) Filed: Feb. 27, 2019

(51) Int. Cl. A61B 5/0205; G01N 27/12; G01N 33/49; A61B 5/00; A61B 5/053; A61B 5/145; A61B 5/0285; A61B 5/0295; H01Q 15/00; H04W 12/06

(52) U.S. Cl. CPC A61B 5/0205; G01N 27/122; G01N 33/492; A61B 5/6862; A61B 5/0031; A61B 5/0538; A61B 5/0215; A61B 5/14542; A61B 5/14546; A61B 5/0285; A61B 5/0295; H01Q 15/0093; H04W 12/06; A61B 5/14532

(57) ABSTRACT

Devices for measuring and communicating physiological data are described. An implanted biocompatible sensor may be utilized to wirelessly communicate with an external transceiver. The biocompatible sensor may include a tubular body, one or more biomedical sensing elements coupled with the tubular body, and a microprocessor in electronic communication with the one or more biomedical sensing elements. The biocompatible sensor may also include an antenna coupled with the tubular body. The transceiver may be in electronic communication with the microprocessor via the antenna and may be configured to power the microprocessor using inductive radio frequency energy.

### BACKGROUND

The following relates generally to medical devices, and more specifically to an injectable biocompatible sensor system for measuring and communicating physiological data.

In a healthcare facility such as a hospital, physiological parameters of the patient (e.g., heart rate, respiratory rate, blood pressure) may be monitored by one or more medical devices. The medical devices may be battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone, tablet, or mobile device).

In some cases, implanting a medical device involves surgically implanting a power source for the medical device within a. patient. Due to the implantable power source and the related surgical time associated with implanting the medical device and power source, patients may be subjected to multiple invasive and time-consuming procedures.

### SUMMARY

The described features generally relate to methods, systems, devices, or apparatuses that support an injectable biocompatible sensor system for measuring and communicating physiological data. A biocompatible sensor is described. The biocompatible sensor may be generally shaped as a stent and include an elongate tubular body configured for insertion in a body lumen. The tubular body may be coupled to one or more biomedical sensory elements configured to measure a characteristic of a fluid flowing through the lumen of the device. In some cases, an antenna is coupled to the device and configured to receive inductive radio frequency energy to power a microprocessor on the medical device. The microprocessor may be in electronic communication with the one or more biomedical sensory elements to measure, process, and communicate physiological data.

A measurement system is described. The measurement system may include the elongate tubular body, the one or more biomedical sensory elements, the microprocessor, the antenna, and a transceiver in electronic communication with the microprocessor via the antenna. In such cases, the transceiver powers the microprocessor using inductive radio frequency energy.

A biocompatible sensor is described. The biocompatible sensor may include an elongate tubular body configured for insertion in a body lumen and comprising a sensor lumen extending from an open proximal end to an open distal end, one or more biomedical sensory elements coupled with the tubular body and configured to measure a characteristic of a fluid flowing through the sensor lumen, a microprocessor in electronic communication with the one or more biomedical sensory elements, and an antenna coupled with an outer surface of the tubular body and configured to receive inductive radio frequency energy to power the microprocessor.

In some examples, the biocompatible sensor may include a communication bus in electronic communication with the one or more biomedical sensory elements and the antenna, wherein the communication bus is configured to transmit power from the antenna to the one or more biomedical sensory elements. In some cases, the communication bus is wrapped at least partially around the outer surface of the tubular body. in some examples, the one or more biomedical sensory elements are each coupled to one or more terminals of the communication bus. In some cases, the antenna is wrapped at least partially around the outer surface of the tubular body. In some cases, the antenna comprises a fractal antenna.

In some examples, the one or more biomedical sensory elements are configured to measure the characteristic of the fluid based at least in part on a change in electrical resistivity of the one or more biomedical sensory elements. In some cases, the one or more biomedical sensory elements comprise one or more carbon nanotube reeds. in some examples, the one or more biomedical sensory elements extend from an inner surface of the tubular body and are configured to bend in response to the fluid flowing through the sensor lumen. In some cases, the one or more biomedical sensory elements are coated with a protein inhibiting material. In some examples, the microprocessor is programmable to program a measuring profile for a measurement of the characteristic of the fluid.

In some examples, the measuring profile comprises a sampling rate, a duration of time to measure the characteristic, a type of the characteristic of the fluid, a. quality command to determine a measurement quality of the one or more biomedical sensory elements, a termination command to deactivate the one or more biomedical sensory elements, or a combination thereof. In some examples, the characteristic of the fluid flowing through the sensor lumen comprises a blood glucose measurement, a blood flow measurement, a blood oxygen concentration measurement, a blood pressure measurement, a blood velocity measurement, a blood sodium concentration measurement, a blood potassium concentration, a blood urea nitrogen (BUN) measurement, or a combination thereof. In some cases, the biocompatible sensor is a medical sensor.

A measurement system is described. The measurement system may include an elongate tubular body configured for insertion in a body lumen, one or more biomedical sensory elements coupled with the tubular body and configured to measure a characteristic of a fluid flowing through the tubular body, a microprocessor in electronic communication with the one or more biomedical sensory elements, an antenna. coupled with an outer surface of the tubular body, and a transceiver in wireless communication with the microprocessor via the antenna and configured to power the microprocessor using inductive radio frequency energy.

In some cases, the microprocessor comprises one or more control logic instructions configured to process a measurement associated with the characteristic of the fluid flowing through a sensor lumen of the tubular body. In some examples, the measurement system may include a communication bus in electronic communication with the one or more biomedical sensory elements and the microprocessor, wherein the communication bus is configured to convey signals from the one or more biomedical sensory elements to the microprocessor. In some cases, the communication bus is wrapped at least partially around the outer surface of the tubular body, and wherein the one or more biomedical sensory elements are each coupled to one or more terminals of the communication bus.

In some examples, the antenna comprises a fractal antenna wrapped at least partially around the outer surface of the tubular body. In some cases, the antenna is configured to receive inductive radio frequency energy to power the microprocessor. In some examples, the transceiver comprises an authentication module configured to authenticate wireless communication between the transceiver and the microprocessor. In some cases, the authentication module is configured to determine that the transceiver is within a proximity threshold of the microprocessor.

In some cases, wireless communication between the transceiver and the microprocessor comprises radiofrequency energy, Bluetooth, Bluetooth low energy (BLE), ultrasonic energy, IR (infrared) energy, wireless local area network (WLAN) computing communications, or a combination thereof. In some examples, the one or more biomedical sensory elements are configured to measure the characteristic of the fluid based at least in part on a change in electrical resistivity of the one or more biomedical sensory elements.

In some examples, the one or more biomedical sensory elements comprise one or more carbon nanotube reeds. In some cases, the one or more biomedical sensory elements extend from an inner surface of the tubular body. In some cases, the one or more biomedical sensory elements are configured to bend in response to the fluid flowing through a sensor lumen extending from an open proximal end to an open distal end of the tubular body. In some examples, the microprocessor is configured to program a measuring profile for a measurement of the characteristic of the fluid.

In some cases, the measuring profile comprises a sampling rate, a duration of time to measure the characteristic, a type of the characteristic of the fluid, a quality command to determine a measurement quality of the one or more biomedical sensory elements, a termination command to deactivate the one or more biomedical sensory elements, or a combination thereof. In some examples, the characteristic of the fluid flowing through the tubular body comprises a blood glucose measurement, a blood flow measurement, a blood oxygen concentration measurement, a blood pressure measurement, a blood velocity measurement, a blood sodium concentration measurement, a blood potassium concentration, a blood urea nitrogen (BUN) measurement, or a combination thereof.

### BRIEF DESCRIPTION OF THE DRAWINGS

FIG. 1 illustrates an example of a wireless patient monitoring system including an injectable biocompatible sensor system for measuring and communicating physiological data in accordance with aspects of the present disclosure.

FIG. 2 illustrates an example of an injectable biocompatible sensor system for measuring and communicating physiological data in accordance with aspects of the present disclosure.

FIG. 3 illustrates an example of an injectable biocompatible sensor system for measuring and communicating physiological data within a body lumen in accordance with aspects of the present disclosure.

FIG. 4A illustrates an example of a top view of an injectable biocompatible sensor system for measuring and communicating physiological data in accordance with aspects of the present disclosure.

FIG. 4B illustrates an example of a bottom view of an injectable biocompatible sensor system for measuring and communicating physiological data in accordance with aspects of the present disclosure.

FIG. 4C illustrates an example of a perspective view of an injectable biocompatible sensor system for measuring and communicating physiological data in accordance with aspects of the present disclosure.

FIG. 4D illustrates an example of a side view of an injectable biocompatible sensor system for measuring and communicating physiological data in accordance with aspects of the present disclosure.

FIG. 5 illustrates an example of a block diagram of an injectable biocompatible sensor system for measuring and communicating physiological data in accordance with aspects of the present disclosure.

### DETAILED DESCRIPTION

An implantable biocompatible device may be used to measure physiological data from within a patient. For example, a biocompatible sensing device may be located within the patient's body and may include a local power source. The biocompatible device may communicate with an external controller via a wireless communication link. To implant a biocompatible sensing device within a patient's body, the patient may undergo multiple medical procedures. For example, a patient may undergo a procedure to implant one or more biocompatible sensing devices and may undergo a subsequent procedure to implant a separate transceiver to communicate the sensed medical data to an external controller. In some cases, the patient undergoes yet another procedure to replace the biocompatible device or recharge the device when a power failure otherwise occurs. Thus an implantable device without a local power source capable of both sensing and communicating the sensed medical data to an external transceiver may reduce an amount of procedures that a patient is subjected to. By reducing a number of procedures that a patient is subjected to, a number of risk factors associated with each unperformed procedure may also be reduced.

In order to reduce the number of devices implanted within a patient's body, a biocompatible sensing device may be implanted within a patient that is in electronic communication with an external transceiver. The biocompatible sensing device may be stent-like in shape and configuration and able to interface with the external transceiver via inductive radio frequency energy. The transceiver may charge the biocompatible sensing device and allow the biocompatible sensing device to collect physiological data. The physiological data may be an example of a characteristic of a fluid flowing through the body lumen (and through a lumen of the biocompatible sensing device). The medial sensing device may collect data for an extended period of time without the need for invasive monitoring and sensing methods. The biocompatible sensing device may provide a platform for various sensors to monitor blood flow, blood pressure, blood velocity, temperature, oxygen levels, ejection fraction, blood glucose, or a combination thereof.

The biocompatible sensing device may be without an internal power source or power storage ability, thereby inductively powered and controlled via the transceiver through near-field communications (NFC), near-field magnetic induction (NFMI), or similar technology. The medial sensing device may include one or more biomedical sensory elements coupled with a tubular body of the biocompatible sensing device. The one or more biomedical sensory elements may measure a characteristic of the fluid flowing through the lumen of the biocompatible sensing device. The biocompatible sensing device may also include a microprocessor in electronic communication with the one or more biomedical sensory elements and an antenna coupled with an outer surface of the biocompatible sensing device. The transceiver may be in electronic communication with the microprocessor via the antenna and may be configured to power the microprocessor using inductive radio frequency energy.

The antenna may be an example of a fractal antenna. The fractal antenna may be an antenna that uses a fractal, self-similar design to maximize the length, or increase the perimeter of material that can receive or transmit electromagnetic radiation within a given total surface area or volume. Fractal antennas may be compact in size, and may utilize multiband and/or wideband signaling. Compared with traditional antenna designs, a fractal antenna may be capable of operating at a high level on a multitude of frequencies simultaneously. Accordingly, fractal antennas may be smaller in size than traditional antennas, yet still provide optimum performance. The reduction in size (e.g., as compared with traditional antenna designs) may allow for a fractal antenna to be implanted within a patient using less-intrusive surgical methods.

Aspects of the disclosure are initially described in the context of a wireless patient monitoring system. Aspects of the disclosure are further illustrated by and described with reference to apparatus diagrams, system diagrams, and flowcharts that relate to injectable sensor system for measuring and communicating physiological data.

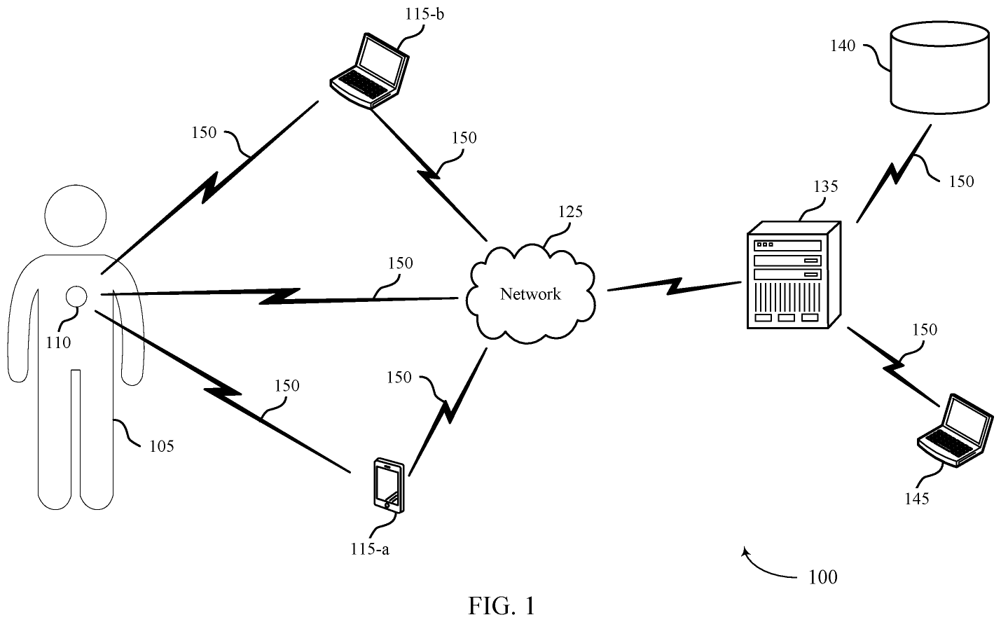

FIG. 1 illustrates an example of a wireless patient monitoring system 100 including an injectable biocompatible sensor system for measuring and communicating physiological data in accordance with aspects of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125.

The medical device 110 may be an example of an injectable biocompatible sensor system as described herein. The medical device 110 may include one or more biomedical sensory elements configured to monitor biometric parameters associated with the patient 105. For example, the one or more biomedical sensory elements may be able to monitor biometric information such as the patient's heart rate, blood pressure, blood glucose, blood velocity, blood flow (e.g., turbulent or laminar flow), blood oxygen concentration, or a combination thereof. In some cases, the medical device 110 includes one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105.

The medical device 110 may be coupled with the patient 105 in a variety of ways depending on the data being collected. In some examples, the medical device 110 is an injectable biocompatible sensor configured for insertion in a body lumen (e.g., a blood vessel) of the patient 105. In such cases, the medical device 110 may be configured to measure a characteristic of a fluid flowing through the body lumen of the patient 105. In some cases, the medical device 110 is externally coupled with the patient 105 (e.g., physically connected to the patient's chest, worn around the patient's wrist or ankle, attached to the patient's finger, or positioned over the patient's nose, mouth, or ear).

The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), infrared (IR) communications, or NFMI),local (e.g., wireless local area network (WLAN)), or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.

Computing device 115-a may be a wireless device such as a tablet, cellular phone, personal digital assistant (PDA), or mobile device, a dedicated receiver (or other similar device(s)) or a spatially distributed network of devices configured to receive signals from the medical device 110. Computing device 115-b may be a wireless laptop computer, a clinician Workstation on Wheels, or a smart hospital bed configured to receive signals from the medical device 110. The computing devices 115 may be in communication with a central station 135 via network 125. In some examples, the computing device 115-a may be an example of a transceiver configured to power the microprocessor (e.g., the medical device 110) using inductive radio frequency energy. The computing device 115-a may be an example of an external power source, a continuous blood glucose monitor transceiver, a NFC or a NFMI enabled diabetic insulin pump, an external heart rate monitor, a ventilator, a NFC or a NFMI enabled device, or a combination thereof. The transceiver may monitor or control the medial device 110 by providing low power communications to the medical device.

The medical device 110 may also communicate directly with the central station 135 via the network 125. The central station 135 may be a server or a central nurse station located within the hospital or in a remote location. The central station 135 may be in further secured communication with one or more remote computing devices 145, thereby allowing a clinician to remotely monitor the patient 105. The central station 135 may also be in secured communication with various remote databases 140 where the collected patient data may be stored. In some cases, the remote databases 140 include electronic medical/health records applications for storing and sharing patient data.

In some examples, the computing device 115-a communicates directly with the medical device 110 associated with the patient 105. The medical device 110 may be directly coupled with (e.g., implanted within) the patient 105, and in some cases, may communicate with the computing device 115-a using inductive radio frequency energy. As described herein, the medical device 110 may include one or more biomedical sensory elements configured to measure physiological data. In some examples, the computing device 115-a is operated by the patient 105 and may securely communicate with the medical device 110 via an antenna (not shown). For example, the antenna may be coupled to the medical device 110 and configured to receive inductive radio frequency energy to power a microprocessor of the medical device 110. The computing device 115-a may transmit, in a secure manner, a signal to the antenna, which may activate and power the microprocessor of the medical device 110 associated with the patient 105.

As described herein, the use of an antenna coupled with the medical device 110 may reduce a total amount of surgical time that the patient 105 is subjected to. For example, the antenna may be located subdermally (e.g., implanted within the patient 105) and may be coupled with the medical device 110. The antenna may be in communication with a transceiver that is external to the patient 105 (e.g., not implanted within the patient 105) and may communicate one or more signals relating to a characteristic of fluid flowing through the body lumen that the one or more biomedical sensory elements are configured to measure. Because the external transceiver is configured to power the antenna, the patient 105 may avoid additional surgeries to implant and replace a power source. In addition, because the transceiver is located external to the patient 105, the patient 105 may avoid implantation of a hazardous power source e.g., batteries, capacitors, or both) configured to store energy within the patient 105. In some examples, the size and orientation of the antenna (e.g., a smaller size as compared with traditional antenna designs) allows for a fractal antenna to be implanted within a patient using less-intrusive surgical methods. In some cases, the orientation of the fractal antenna does not impede the function of the antenna due to the omnidirectional properties of the antenna.

In some examples, the use of an antenna coupled with the one or more biomedical sensory elements replaces existing methods for device to device (D2D) data bridging and intrabody signals transmission by receiving communications external to the patient 105 and implementing them using an implanted medical device 110. In some examples, the system includes one or more low power bio-inert fractal antennas and an embedded programmable processing module that may enable implanted medical devices to transmit device and/or sensor signals within a biological environment. Thus, in addition to reducing a number of intrabody devices and surgical procedures, the use of a bio-inert fractal antenna coupled with one or more biomedical sensory elements may provide for more effective medical operations and patient monitoring.

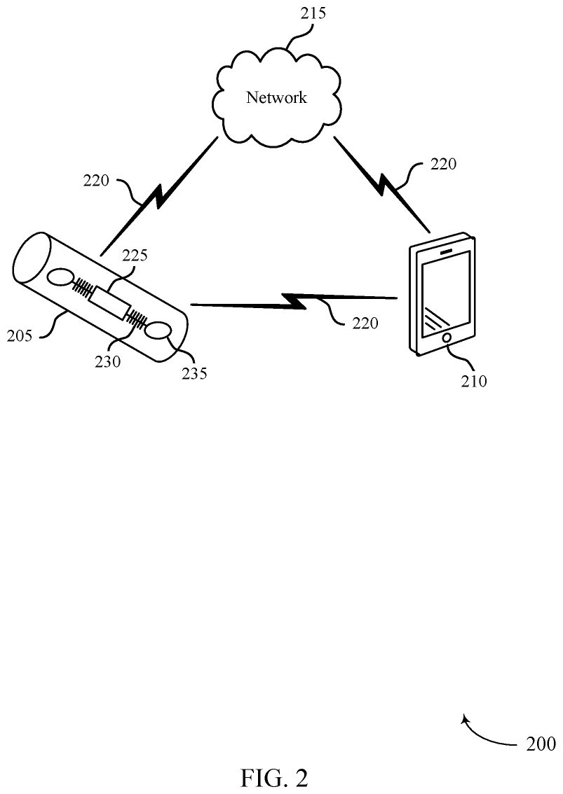

FIG. 2 illustrates an example of an injectable biocompatible sensor system 200 for measuring and communicating physiological data in accordance with aspects of the present disclosure. The medical measurement system 200 may be an example of aspects of the system 100 and may include a biocompatible sensor 205 which may be an example of medical device 110 as described with reference to FIG. 1, in such cases, biocompatible sensor 205 may be a medical sensor. In some examples, the medical measurement system 200 includes a transceiver 210, which may be an example of computing device 115-a or 115-b as described with reference to FIG. 1, and a network 215, which may be an example of network 125 as described with reference to FIG. 1. Each of the components illustrated may be connected via communication links 220, which may be examples of communication links 150 as described with reference to FIG. 1.

In some examples, the biocompatible sensor 205 includes a microprocessor 225, an antenna 230, and one or more biomedical sensory elements 235. As discussed below in further detail, the biocompatible sensor 205 may include a tubular body configured for insertion in a body lumen (e.g., a blood vessel). The one or more biomedical sensory elements 235 may be coupled with the tubular body of the biocompatible sensor 205 and configured to measure a characteristic of a fluid flowing through the lumen of the biocompatible sensor 205. In such cases, the biomedical sensory elements 235 may collect sensory data For example, the biomedical sensory elements 235 may measure a change in resistance of the biomedical sensory elements 235 as the fluid flows through the lumen of the biocompatible sensor 205. The collected sensory data may be conveyed to the microprocessor 225 which may be in electronic communication with the one or more biomedical sensory elements 235. The microprocessor 225 may process the sensory data and transmit the processed sensory data securely to the transceiver 210 via the antenna 230.

In some cases, the antenna 230 is coupled to the tubular body of the biocompatible sensor 205 and configured to receive inductive radio frequency energy. The inductive radio frequency energy may passively power the microprocessor 225. For example, the microprocessor 225 may be without power and may be initially charged using inductive radio frequency energy. Thus, the patient may be subject to fewer medical procedures (e.g., as compared to a sensor with an internal power source) when the biocompatible sensor 205 requires a recharge. That is, the biocompatible sensor 205 may be powered while remaining within the body lumen of the patient.

The antenna 230 may be coupled with an outer surface of the biocompatible sensor 205. In some cases, the transceiver 210 is in wireless communication with the microprocessor 225 via the antenna 230. The transceiver 210 may be configured to power the microprocessor 225 using inductive radio frequency energy.

The transceiver 210 may be configured to communicate with the microprocessor 225 and wirelessly transmit a signal (e.g., control information) relating to a characteristic of fluid flowing through the body lumen of the patient to the microprocessor 225. In such cases, the signal may indicate a particular characteristic of the fluid to be measured. The wireless communication between the transceiver 210 and the microprocessor 225 may include radiofrequency energy, NFMI, Bluetooth BLE, ultrasonic energy, IR energy, WLAN computing communications, or a combination thereof.

Because the transceiver 210 is located external to the patient, the patient may be subjected to fewer medical procedures (e.g., as compared with an internal transceiver 210). The antenna 230 may be coupled with the one or more biomedical sensory elements 235, which are both implanted within the patient with the biocompatible sensor 205. The antenna 230 may receive one or more signals (e.g., an inductive radiofrequency signal) from the transceiver 210 (e.g., via communication link 220 or via communication link 220 by way of network 215) and subsequently transmit a signal to the one or more biomedical sensory elements 235 via the microprocessor 225. As described in more detail below, these signals from the transceiver 210 may be converted to power for the microprocessor 225 and the other electrical components of the biocompatible sensor 205. Due to the one or more signals being received from a device located external to the patient (e.g., from transceiver 210), the biocompatible sensor 205 may not need an implanted power source. Accordingly, the patient may be subjected to fewer medical procedures as compared to a patient with an implanted power source. The patient with an implanted power source may be subject to multiple procedures when the power source requires a recharge or exchange, Thus, the patient without an implanted power source may be subject to fewer medical complications than a patient with an implanted power source.

In some cases, the transceiver 210 authenticates wireless communication between the transceiver 210 and the microprocessor 225. For example, if a user of the transceiver 210 possesses the requisite credentials, the transceiver 210 may authenticate the microprocessor 225 upon receiving a signal from the microprocessor 225. In some cases, the transceiver 210 includes an authentication module to authenticate the microprocessor 225. A transceiver 210 may be required to possess credentials in order for the physiological data to be measured and communicated from the biocompatible sensor 205 to the transceiver 210. As described herein, the transceiver 210 may authenticate the microprocessor based on one or more credentials of the user and/or a characteristic of the signal received. The signal may be transmitted directly to the transceiver via communication link 220 or, in other examples, may be transmitted via communication link 220 by way of network 215. Data transmitted via the communication link 220 may be encrypted. For example, data transmitted from the transceiver 210 to the network 215 may be encrypted and, data transmitted from the antenna 230 to the transceiver 210 may be encrypted.

In some cases, the transceiver 210 is configured to determine that the transceiver 210 is within a proximity threshold of the microprocessor 225. The transceiver 210 may authenticate wireless communication between the transceiver 210 and the microprocessor 225 based on determining that the transceiver 210 is within the proximity threshold. For example, the transceiver 210 may communicate with the microprocessor 225 within the proximity threshold, and the transceiver 210 may halt communications with the microprocessor 225 if the transceiver 210 exceeds the proximity threshold. The authentication module of the transceiver 210 may increase the security of the data transmission between the transceiver 210 and the biocompatible sensor 205 to eliminate signal interface, bluejacking issues, or both. The authentication module may be an example of an encryption module that reduces risks of exploitation that the biocompatible sensor 205 may encounter. The authentication module may enable secure communications.

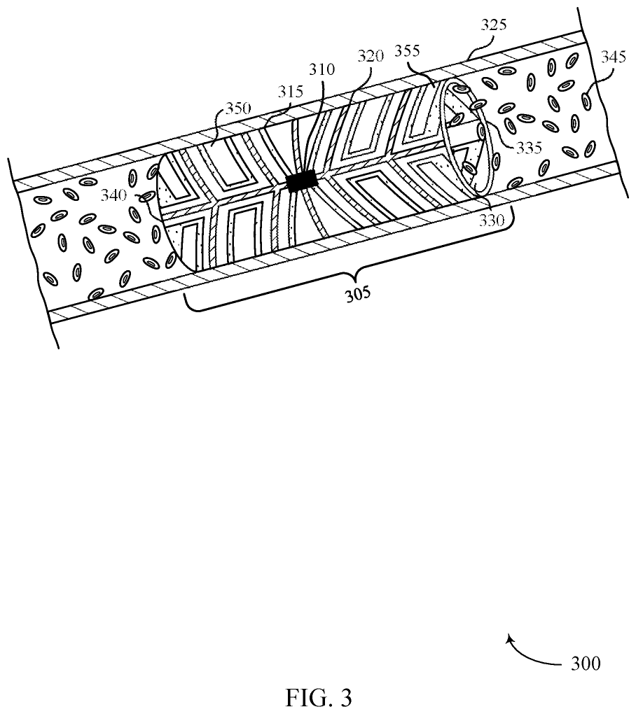

FIG. 3 illustrates an example of an injectable biocompatible sensor system for measuring and communicating physiological data within a body lumen in accordance with aspects of the present disclosure. In some examples, the system 300 includes a biocompatible sensor 305, a microprocessor 310, an antenna 315, and a communication bus 320. The biocompatible sensor 305 may be implanted within a body lumen 325 (e.g., a blood vessel). The antenna 315 may be or may be referred to as a fractal antenna and may be an example of antenna 230 as described with reference to FIG. 2. In some examples, the biocompatible sensor 305 and the microprocessor 310 may be an example of biocompatible sensor 205 and microprocessor 225, respectively, as described with reference to FIG. 2. In some examples, the microprocessor 310 communicates with a transceiver (not shown) that may be associated with, for example, a user, a clinician, or both.

The biocompatible sensor 305 may include a lumen 330 that extends from an open proximal end 335 to an open distal end 340. Fluid 345 may flow through the lumen 330 of the biocompatible sensor 305. In some examples, the fluid 345 is blood. However, the biocompatible sensor 305 may be configured and sized for implantation into body lumens other than a blood vessel (e.g., lumens of the biliary, urinary, gastrointestinal, or bronchial systems). The biocompatible sensor 305 may also include an outer surface 350. The outer surface 350 of the biocompatible sensor 305 may be adjacent to an inner surface of the body lumen 325 such that the biocompatible sensor 305 is coaxial with the body lumen 325. That is, the fluid 345 may flow through the lumen 330 of the biocompatible sensor 305 (and through body lumen 325) without obstruction. In some examples, the biocompatible sensor 305 is a biocompatible stent or stent-like device. The biocompatible stent or stent-like device may include a length of 4 mm and a diameter of 0.75 mm. The length and diameter of the biocompatible stent or stent-like device is not limited to the examples and designs described herein. The biocompatible sensor 305 may conform to size and shape of the body lumen 325 when the biocompatible sensor 305 is in an expanded state.

The biocompatible sensor 305 may also include the communication bus 320. The communication bus 320 may be in electronic communication with the one or more biomedical sensory elements (not shown) and the antenna 315. The antenna 315 may be coupled with the outer surface 350 of the tubular body 355. As described below in further detail, the communication bus 320 may be configured to securely transmit power and signaling between the antenna 315 and the one or more biomedical sensory elements.

The biocompatible sensor 305 may be implanted subcutaneously within the patient (e.g., within the body lumen 325) and may communicate with a transceiver (not shown) that is located external to the patient. The biocompatible sensor 305 can be configured for implantation in any location of the patient's body. For example, the biocompatible sensor 305 may be implanted within the smaller muscular arteries (e.g., sub-dermis). The antenna 315 may receive one or more signals (e.g., an inductive radiofrequency signal or NFMI) from the transceiver and subsequently transmit a signal to the one or more biomedical sensory elements via the microprocessor 310. For example, a transceiver may transmit a signal for measuring a characteristic of the fluid 345 flowing through the body lumen 325 to the microprocessor 310. The antenna 315 may, in some examples, generate a signal and transmit the signal to the one or more biomedical sensory elements. As described herein, the signal transmitted to the one or more biomedical sensory elements may, in some examples, result in the one or more biomedical sensory elements measuring a characteristic of the fluid 345 flowing through the lumen 330 of the biocompatible sensor 305.

In some examples, the antenna 315 is configured to receive a signal (e.g., from a user device associated with a user, a clinician, or both) from the transceiver. Thus, upon receiving the signal, the antenna 315 may transmit the signal to the microprocessor 310. The microprocessor 310 may be configured to activate the one or more biomedical sensory elements. In some examples, the signal indicates a particular characteristic of the fluid 345 to be measured. However, in some examples, the signal includes one or more characteristics of the fluid 345 to be measured. Thus, when received by the antenna 315, the microprocessor 310 may determine the particular characteristic to measure of the fluid 345 (e.g., via the one or more biomedical sensory elements).

The one or more biomedical sensory elements may be configured to monitor biometric parameters associated with the patient. For example, the one or more biomedical sensory elements may be able to monitor biometric information such as the patient's heart rate, blood pressure, blood glucose, blood velocity, blood flow (e.g., turbulent or laminar flow), blood oxygen concentration, or a combination thereof. In some cases, the blood velocity measurement determines a vascular occlusion. In some examples, biometric information includes blood sodium concentration, blood potassium concentration, blood urea nitrogen (BUN) concentration, or a combination thereof. In other examples, biometric information may include an ejection fraction (EF) measurement, detecting diabetic ketoacidosis (DKA), detecting decreased kidney function, detecting renal failure, determining a platelet count, determining a core body temperature, or a combination thereof.

Because the antenna 315 may be configured to receive a secure signal from the transceiver and subsequently activate (e.g., via the microprocessor 310) the one or more biomedical sensory elements in order to measure a characteristic of fluid flowing through the body lumen 325 of the patient, the antenna 315 may be located anywhere within the patient. By implanting the biocompatible sensor 305 within the body lumen 325 of the patient, the patient may be subjected to fewer operations to implant medical devices. For example, implanting a power source within the patient may subject the patient to additional surgical procedures if the power source diminishes.

As described herein, because the transceiver is located external to the patient yet still able to communicate with the antenna 315 and microprocessor 310, the patient may experience fewer (e.g., as compared with an internal transceiver) implantation procedures. Further, fewer medical devices located within a patient may result in reduced failure rates related to breakage, impedance, maintenance requirements, and may reduce patient complications due to infection resulting from the implantation of various devices. In addition, implanting the biocompatible sensor 305 within the patient may eliminate sensor damage associated with physical activity, handling, impact damage, or a combination thereof such as may occur with use of an external biocompatible sensor 305.

For example, a blood glucose sensing device (e.g., external biocompatible sensor 305) may be located on a surface of the patient's body and may be coupled with a diabetic insulin pump (e.g., external transceiver). The transceiver may communicate with the external biocompatible sensor 305 via wireless communication. However, the external biocompatible sensor 305 may be subject to relocation, multiple medical procedures for relocation, adverse sensor attachment methods, or sensor damage (e.g., physical activity or handling). Thus a system capable of implanting a biocompatible sensor 305 within the patient's body to communicate with an external transceiver may reduce the risk of decreased sensor performance and multiple medical procedures. The patient may also experience fewer complications associated with relocating the biocompatible sensor 305 within the patient as well as sensor attachment methods associated with an external biocompatible sensor 305.

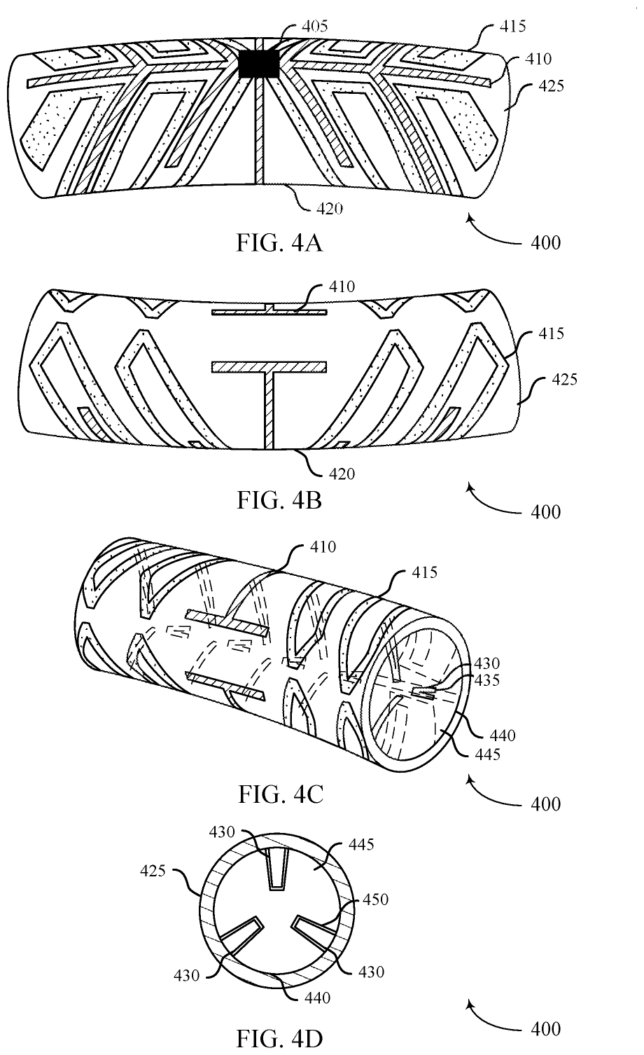

FIG. 4A illustrates an example of a top view of an injectable biocompatible sensor system for measuring and communicating physiological data in accordance with aspects of the present disclosure. The biocompatible sensor 400 may include a microprocessor 405, a communication bus 410, and an antenna 415. In some examples, the microprocessor 405, the communication bus 410, and the antenna 415 may each be an example of the microprocessor, the communication bus, and the antenna as described with reference to FIGS. 2 and 3.

The biocompatible sensor 400 includes a tubular body 425 that comprises an outer surface 420. The antenna 415 may be coupled with the outer surface 420 of the tubular body 425 and configured to receive inductive radio frequency energy or NFMI to power the microprocessor 405. The antenna 415 may include a self-similar design to increase the perimeter of material that can receive or transmit signals (e.g., an inductive radio frequency signal or NFMI). This may result in the antenna 415 being both compact and wideband. In some examples, the antenna 415 includes a flexible (e.g., an adaptable) material. The flexibility of the antenna 415 may allow it to, for example, conform to the curvature of the outer surface 420 of the tubular body 425. In such cases, the antenna 415 is wrapped at least partially around the outer surface 420 of the tubular body 425.

In some examples, the antenna 415 supports communication protocols that use wider (e.g., as compared with a traditional antenna) frequency ranges. For example, the antenna 415 supports multiple protocols such as 802.11ax or future advanced protocols in order to provide higher data throughput and lower latency. In some examples, the antenna 415 operates in less-crowded spectrum bands by operating in the 60 GHz millimeter wave band. The 60 GHz spectrum band may accommodate ultra-wideband channels that enable multiple Gigabit-per-second data rates.

In some cases, the antenna 415 comprises a fractal antenna. The properties of the antenna 415 may allow for it to have many different resonances, meaning it will act as an antenna for many different electromagnetic frequencies. In some examples, the different resonances results from a portion of the antenna 415 acting as a virtual network of capacitors and a different portion of the antenna 415 acting as a virtual network of inductors. The antenna 415 differs from traditional antenna designs (e.g., non-fractal antennas), in that the antenna 415 may be capable of operating with good-to-excellent performance at many different frequencies simultaneously. Standard antennas (e.g., non-fractal antennas) may have to be “cut” for the frequency for which they are to be operated and thus the standard antennas only work well at one particular frequency.

In some examples, the antenna 415 receives one or more signals from an external device (e.g., a transceiver). The signals may be received by the antenna 415 and transmitted to the microprocessor 405, which may include one or more processors. In some examples, the microprocessor 405 is configured to receive and/or process a signal received by the antenna 415. The signal may, as described herein, relate to a characteristic of a fluid flowing through the lumen to be measured by the biocompatible sensor 400-a. In some examples, the signal includes identifying characteristics that may be used for authentication purposes.

As described herein, the microprocessor 405 may be programmable to program a measuring profile for a measurement of the characteristic of the fluid. For example, the measuring profile may be an example of a sampling rate, a duration of time to measure the characteristic, a type of the characteristic of the fluid. In some examples, the measuring profile is an example of a quality command to determine a measurement quality of the one or more biomedical sensory elements 430, a termination command to deactivate the one or more biomedical sensory elements 430, or a combination thereof.

In some examples, the microprocessor 405 includes a radio frequency chip and/or a transmitter for transmitting one or more secure signals via the antenna 415. The microprocessor 405 may include a radiofrequency chip such that, if the antenna 415 receives a radiofrequency signal, the microprocessor 405 may be configured to decipher the signal. Additionally or alternatively, the radiofrequency chip may be configured to convert the received radiofrequency signal into a power or energy source to power the antenna 415. In some examples, the microprocessor 405 includes a transmitter in order to transmit one or more signals via the antenna 415.

In some examples, the antenna 415 is fabricated at a nano-scale. The antenna 415 may be fabricated as or on a relatively small circuit board (e.g., a nano-sized circuit board) to facilitate implantation into the body (e.g., within a body lumen). The relatively small size of the antenna 415 may, for example, allow versatility in its use with other wireless devices. For example, in a medical environment as described herein, the antenna 415 may support Digital Signal Processing (DSP). Accordingly, the antenna 415 may be fabricated on a dedicated ASIC, FPGA, GPU, and/or DSP chip, and/or may include an embedded processor with DSP extensions. In some examples, the antenna 415 includes a SoC (System-on-Chip) architecture that is based on a FPGA (Field-Programmable Gate Array) medical device solution (e.g., that is suitable for the implementation of biomedical signal processing).

FIG. 4B illustrates an example of a bottom view of an injectable biocompatible sensor system for measuring and communicating physiological data in accordance with aspects of the present disclosure. The communication bus 410 and the antenna 415 are each wrapped at least partially around the outer surface 420 of the tubular body 425.

FIG. 4C illustrates an example of a perspective view of an injectable biocompatible sensor system for measuring and communicating physiological data in accordance with aspects of the present disclosure. The biocompatible sensor 400 may include biomedical sensory elements 430. In some examples, the biomedical sensory elements may be an example of the biomedical sensory elements as described with reference to FIG. 2.

The communication bus 410 may be in secure electronic communication with the one or more biomedical sensory elements 430 and the antenna 415. The communication bus 410 may be configured to transmit power and signaling (e.g., control and data signaling) between the antenna 415 and the one or more biomedical sensory elements 430. The biomedical sensory elements 430 may be configured to measure the characteristic of the fluid based at least in part on a change in electrical resistivity of the one or more biomedical sensory elements 430. For example, the change in electrical resistivity may increase within the biomedical sensory elements 430 when a bending stress at compressive and tensile points of the biomedical sensory elements 430 increase.

In some cases, the microprocessor 405 authenticates the identifying characteristics of the fluid such that the one or more biomedical sensory elements 430 may deliver a resulting electrical or electromagnetic signal to the patient. The microprocessor 405 may also power the biomedical sensory elements 430. In some cases, the microprocessor 405 powers each individual biomedical sensory element 430 according to a timing circuit embedded within the microprocessor 405. In other examples, the microprocessor 405 powers the biomedical sensory elements 430 at a same time. The biomedical sensory elements 430 may each be coupled to one or more terminals 435 of the communication bus 410. For example, the biomedical sensory elements 430 may include a staggered configuration throughout the inner surface 440 of the lumen based on the location of the one or more terminals 435.

As described herein, the biomedical sensory elements 430 may be configured to measure biometric information related to a patient (e.g., due to being implanted within a patient). The biomedical sensory elements 430 may transmit the biometric information to the microprocessor 405 (e.g., to the transmitter), which may process the data according to a programmed measuring profile. The microprocessor 405 may also transmit the information via the antenna 415. In some examples, the biometric information is transmitted (e.g., using the antenna 415) to an external transceiver e.g., a user device associated with the patient).

FIG. 4D illustrates an example of a side view of an injectable biocompatible sensor system for measuring and communicating physiological data in accordance with aspects of the present disclosure. In some examples, the biomedical sensory elements 430 include or are made from one or more carbon nanotube reeds. The biomedical sensory elements 430 may extend from an inner surface 440 of the tubular body 425 into the lumen 445 of the tubular body 425. For example, the biomedical sensory elements 430 may extend in a direction perpendicular to the inner surface 440 of the tubular body 425. Thus, the biomedical sensory elements 430 may be designed and configured to bend in response to the fluid flowing through the lumen 445.

The sensing platform (e.g., biomedical sensory elements 430) may not obstruct fluid flow through the lumen 445 (and therefore through the body lumen itself). The biomedical sensory elements 430 may directly contact the fluid flowing through the lumen 445 to directly sample the characteristic of the blood rather than extrapolation from a tissue surface. Thus, the measurements by the biomedical sensory elements 430 may be more reliable than a measurement extrapolated from a tissue surface or sample.

The biomedical sensory elements 430 may bend based on a characteristic of the fluid flowing through the lumen 445. Each biomedical sensory element 430, composed of carbon nanotubes fluid-flow reed sensors (e.g. when the fluid may be blood), may deflect the fluid at different angles. The degree of deflection for each carbon nanotube reed may enable the microprocessor to calculate the desired measurement and extrapolate the sensory data based on the reed deflection. In some cases, the extrapolation determines a vascular occlusion (e.g., impediment upstream in the body lumen) based on the measured blood velocity or blood pressure.

In some cases, the biomedical sensory elements 430 are coated with a protein inhibiting material 450. For example, the protein inhibiting material 450 may be an example of an immune system inhibitor to allow the biocompatible sensor 400 to be biocompatible.

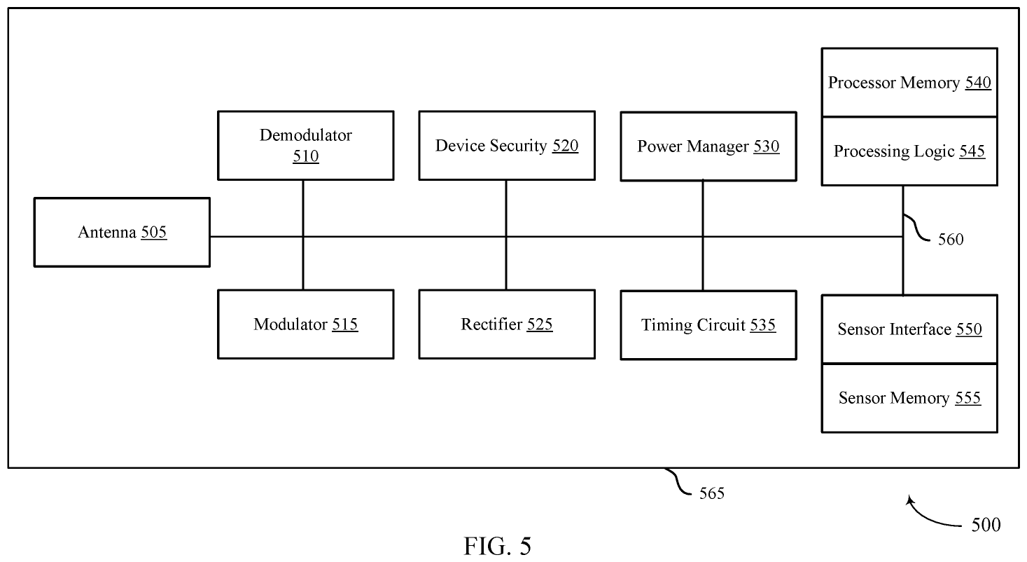

FIG. 5 illustrates an example of a block diagram of an injectable biocompatible sensor system for measuring and communicating physiological data in accordance with aspects of the present disclosure. The system 500 may include an antenna 505, communication bus 560, and a biocompatible sensor 565. In some examples, the antenna 505, the communication bus 560, and the biocompatible sensor 565 may each be an example of the antenna, the communication bus, and the biocompatible sensor as described with reference to FIGS. 2 through 4. The antenna 505 may be in electronic communication with the communication bus 560.

In some cases, system 500 includes one or more modules. The antenna 505 may be in electronic communication with the one or more modules via the communication bus 560. The system 500 may include a demodulator 510 which may be an example of a receiver. The demodulator 510 may detect a command sent by the transceiver. In some cases, the demodulator 510 extracts a clock from the received radiofrequency signal. In such cases, the clock synchronizes a radiofrequency or NFMI transponder with the transceiver.

The system 500 may include a modulator 515. The modulator 515 may be an example of a transmitter of the system 500 that may send the transponder identification and sensor data to the transceiver. In some cases, the system 500 includes device security module 520. The device security module 520 may ensure that filtering between the transponder and the transceiver may restrict and/or control transponder access. In some cases, the device security module 520 regulates the inductive radio frequency energy or the NFMI transmission energy with a located magnetic field near-field).

In some examples, the system 500 includes a rectifier 525. The rectifier 525 may rectify the input radio frequency signal or the NFMI signal and manage the direct current (DC) voltage to power the other modules in the system 500. In some cases, the rectifier 525 generates the DC voltage to power the other modules in the system 500. The rectifier 525 may also include a temperature regulator to regulate the temperature profile of the microprocessor. The system 500 may also include a power manager 530. The power manager 530 may regulate power to the control circuits, sensors (e.g., biomedical sensory elements), a data transmission circuit, an antenna, or a combination thereof.

The system 500 may include a timing circuit 535. The timing circuit 535 may be an example of an internal clock that supplies a generated timing signal to the digital elements of the transponder. In such cases, the timing circuit 535 may control the use of multiple timers for establishing data collection duration and sample rates. In some cases, additional timing enables configuration of biomedical sensory element sample rates, hibernation states, transmission wake-up (e.g., sample start time, sample transmission time, or both), transmission duration, or a combination thereof.

The system 500 may include a processor memory 540 and a processing logic 545. The processing logic 545 may be an example of a digital component of the system 500 (e.g., microcontroller) that controls the other modules of the system 500. In some examples, the processing logic 545 stores the transponder identification, process device instructions, determine when to collect data., when to transmit data, when to hibernate, or a combination thereof.

In some examples, the processing logic 545 of the microprocessor is programmable to program a measuring profile for a measurement of the characteristic of the fluid. For example, the processing logic 545 may include a sampling rate, a duration of time to measure the characteristic, a type of the characteristic of the fluid, or a combination thereof. The sampling rate may be a continuous sampling rate. The processing logic 545 may include a quality command to determine a measurement quality of the one or more biomedical sensory elements, a termination command to deactivate the one or more biomedical sensory elements, or both. For example, the quality command determines a failure associated with the biomedical sensory element. The failure or reliability of the biomedical sensing element may be due to a protein build up on the carbon nanotube reed where the reed may no longer be able to react to fluid flow. In some cases, the processing logic 545 is an example of one or more control logic instructions configured to process a measurement associated with the characteristic of the fluid flowing through a sensor lumen of the tubular body.

In some cases, the system 500 includes a sensor interface 550 and a sensor memory 555. The sensor interface 550 may control sensor activation, sensor sample timing, results collection, or a combination thereof for the biomedical sensory elements. The transponder may communicate via inductive or magnetic coupling by operating in the high frequency of 13.56 MHz wave band. The transponder frequency may comply with standards classified worldwide as ISM (industrial, scientific, medical) frequency ranges for use in ISM environments or by short-range devices (SRD) frequency.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A biocompatible sensor, comprising:
an elongate tubular body configured for insertion in a body lumen and comprising a sensor lumen extending from an open proximal end to an open distal end;
one or more biomedical sensory elements coupled with the tubular body and configured to measure a characteristic of a fluid flowing through the sensor lumen;
a microprocessor in electronic communication with the one or more biomedical sensory elements; and
an antenna coupled with an outer surface of the tubular body and configured to receive inductive radio frequency energy to power the microprocessor.

2. The biocompatible sensor of claim 1, further comprising:
a communication bus in electronic communication with the one or more biomedical sensory elements and the antenna, wherein the communication bus is configured to transmit power from the antenna to the one or more biomedical sensory elements.

3. The biocompatible sensor of claim 2, wherein the communication bus is wrapped at least partially around the outer surface of the tubular body.

4. The biocompatible sensor of claim 2, wherein the one or more biomedical sensory elements are each coupled to one or more terminals of the communication bus.

5. The biocompatible sensor of claim 1, wherein the antenna is wrapped at least partially around the outer surface of the tubular body.

6. The biocompatible sensor of claim 1, wherein the antenna comprises a fractal antenna.

7. The biocompatible sensor of claim 1, wherein the one or more biomedical sensory elements are configured to measure the characteristic of the fluid based at least in part on a change in electrical resistivity of the one or more biomedical sensory elements.

8. The biocompatible sensor of claim 1,wherein the one or more biomedical sensory elements comprise one or more carbon nanotube reeds.

9. The biocompatible sensor of claim 1, wherein the one or more biomedical sensory elements extend from an inner surface of the tubular body and are configured to bend in response to the fluid flowing through the sensor lumen.

10. The biocompatible sensor of claim 1, wherein the one or more biomedical sensory elements are coated with a protein inhibiting material.

11. The biocompatible sensor of claim 1, wherein the microprocessor is programmable to program a measuring profile for a measurement of the characteristic of the fluid.

12. The biocompatible sensor of claim 11, wherein the measuring profile comprises a sampling rate, a duration of time to measure the characteristic, a type of the characteristic of the fluid, a quality command to determine a measurement quality of the one or more biomedical sensory elements, a termination command to deactivate the one or more biomedical sensory elements, or a combination thereof.

13. The biocompatible sensor of claim 1, wherein the characteristic of the fluid flowing through the sensor lumen comprises a blood glucose measurement, a blood flow measurement, a blood oxygen concentration measurement, a blood pressure measurement, a blood velocity measurement, a blood sodium concentration measurement, a blood potassium concentration, a blood urea nitrogen (BUN) measurement, or a combination thereof.

14. The biocompatible sensor of claim 1, wherein the biocompatible sensor is a medical sensor.

15. A measurement system, comprising:
an elongate tubular body configured for insertion in a body lumen;
one or more biomedical sensory elements coupled with the tubular body and configured to measure a characteristic of a fluid flowing through the tubular body;
a microprocessor in electronic communication with the one or more biomedical sensory elements;
an antenna coupled with an outer surface of the tubular body; and
a transceiver in wireless communication with the microprocessor via the antenna and configured to power the microprocessor using inductive radio frequency energy.

16. The measurement system of claim 15, wherein the microprocessor comprises one or more control logic instructions configured to process a measurement associated with the characteristic of the fluid flowing through a sensor lumen of the tubular body.

17. The measurement system of claim 15, further comprising:
a communication bus in electronic communication with the one or more biomedical sensory elements and the microprocessor, wherein the communication bus is configured to convey signals from the one or more biomedical sensory elements to the microprocessor.

18. The measurement system of claim 17, wherein the communication bus is wrapped at least partially around the outer surface of the tubular body, and wherein the one or more biomedical sensory elements are each coupled to one or more terminals of the communication bus.

19. The measurement system of claim 15, wherein the antenna comprises a fractal antenna wrapped at least partially around the outer surface of the tubular body.

20. The measurement system of claim 15, wherein the antenna is configured to receive inductive radio frequency energy to power the microprocessor.

21. The measurement system of claim 15, wherein the transceiver comprises an authentication module configured to authenticate wireless communication between the transceiver and the microprocessor.

22. The measurement system of claim 21, wherein the authentication module is configured to determine that the transceiver is within a proximity threshold of the microprocessor.

23. The measurement system of claim 15, wherein:
wireless communication between the transceiver and the microprocessor comprises radiofrequency energy, Bluetooth, Bluetooth low energy (BIT), ultrasonic energy, IR (infrared) energy, wireless local area network (WLAN) computing communications, or a combination thereof.

24. The measurement system of claim 15, wherein the one or more biomedical sensory elements are configured to measure the characteristic of the fluid based at least in part on a change in electrical resistivity of the one or more biomedical sensory elements.

25. The measurement system of claim 15, wherein the one or more biomedical sensory elements comprise one or more carbon nanotube reeds.

26. The measurement system of claim 15, wherein the one or more biomedical sensory elements extend from an inner surface of the tubular body.

27. The measurement system of claim 15, wherein the one or more biomedical sensory elements are configured to bend in response to the fluid flowing through a sensor lumen extending from an open proximal end to an open distal end of the tubular body.

28. The measurement system of claim 15, wherein the microprocessor is configured to program a measuring profile for a measurement of the characteristic of the fluid.

29. The measurement system of claim 28, wherein the measuring profile comprises a sampling rate, a duration of time to measure the characteristic, a type of the characteristic of the fluid, a quality command to determine a measurement quality of the one or more biomedical sensory elements, a termination command to deactivate the one or more biomedical sensory elements, or a combination thereof.

30. The measurement system of claim 15, wherein the characteristic of the fluid flowing through the tubular body comprises a blood glucose measurement, a blood flow measurement, a blood oxygen concentration measurement, a blood pressure measurement, a blood velocity measurement, a blood sodium concentration measurement, a blood potassium concentration, a blood urea nitrogen (BUN) measurement, or a combination thereof.
